(19) United States
(12) Patent Application Publication
(10) Pub. No.: US 2003/0135153 A1
(43) Pub. Date: Jul. 17, 2003

(54) Drug implant injection device

(76) Inventor: Charles Hagemeier, Laguna Beach, CA (US)

Correspondence Address: Bausch & Lomb Incorporated, One Bausch & Lomb Place, Rochester, NY, 14604-2701, US

(21) Appl. No.: 10/309,589

(22) Filed: Dec. 4, 2002

Related U.S. Application Data
(60) Provisional application No. 60/341,739, filed on Dec. 17, 2001.

(51) Int. Cl. A61M 31/00

(52) U.S. Cl. 604/59

(57) ABSTRACT

A device for delivering a drug implant to an implant site comprises a needle having a lateral opening in which the drug implant is releasably held. Once at the implant site, the drug implant is released directly form the opening. The invention obviates the need for a cannula through which a drug implant is delivered, and therefore obviates the many problems associated with a cannula-type delivery device.

### BACKGROUND OF THE INVENTION

The present invention relates to a device and method for delivering a drug directly to an interior portion of a mammalian body. More particularly, the present invention relates to an improved drug injection device and method which is operable to effectively and easily inject a sustained release drug implant into thin tissues, such as the sclera of an eye, for example.

Over the years, various drugs have been developed to assist in the treatment of a wide variety of ailments and diseases. However, in many instances such drugs are not capable of being administered either orally or intravenously without the risk of various detrimental side effects.

CMV retinitis is a disease that is characterized by inflammation of the retina caused by infection with cytomegalovirus. CMV retinitis is one of the most common causes of sight-threatening infections among people with HIV. The symptoms include loss of visual acuity, blind spots, and the loss of peripheral vision. Left untreated, CMV retinitis can lead to blindness.

Intravenous ganciclovir (GCV) is effective in the treatment of CMV retinitis in AIDS patients, but bone marrow toxicity limits its usefulness. Continuous maintenance GCV therapy is necessary to prevent progression or recrudescence of the disease, but despite maintenance therapy a significant number of patients experience a relapse during treatment. Additionally, there are other risks and problems associated with systemic GCV administration.

Intravitreal GCV injections administered once or twice weekly have resulted in temporary remission of CMV retinitis in AIDS patients. Intravitreal GCV injections may provide a higher intraocular drug concentration than systemic therapy and reduce the incidence of neutropenia. However, current treatment of CMV retinitis in AIDS patients is clearly suboptimal. Ganciclovir is virustatic and thus disease inhibition requires maintenance drug administration.

A more detailed explanation of the use of intravenous GCV and intravitreal injections of GCV can be found in U.S. Pat. No. 5,902,598, herein incorporated in its entirety by reference. A discussion of the difficulties associated with the systemic therapy of cyclosporine A in the treatment of uveitis can be found in U.S. Pat. Nos. 5,773,019 and 6,001,386, herein incorporated in their entirety by reference.

Accordingly, there exists a strong need for the elimination of the undesirable physiological problems associated with GCV treatment of CMV retinitis, while maintaining the advantageous properties of this treatment. Although delivering the drug locally with injections may minimize the systemic toxicity of GCV, repeated injection is not a practical mode of administration.

Due to the risks that certain drugs impose, researchers have developed systems for administering such drugs to aid in the treatment of these ailments and diseases. A general discussion of drug delivery control systems is provided in Controlled Drug Delivery (Part I), Xue Shen Wu, Ph.D. pp32, 33, 44-46, 63, 66, and 67 (Technomic Publishing Co. Inc., 1996), the entire contents of which are incorporated herein by reference. The systems have been designed largely to reduce and to control the release rate of incorporated drugs. However, these systems failed to achieve many of the advantages solved by later devices.

For example, U.S. Pat. No. 4,014,335 to Arnold, relates to various ocular inserts that act as a deposit or drug reservoir for slowly releasing a drug into the tear film for prolonged periods of time. These inserts are fabricated as a three-layer laminate of flexible polymeric materials that are biologically inert, non-allergenic, and insoluble in tear fluid. To initiate the therapeutic programs of these devices, the ocular inserts are placed in the cul-de-sac between the sclera of the eyeball and the eyelid for administering the drug to the eye. Multiple layer laminate systems can present a challenge to reproducibly manufacture and are more difficult to produce by large-scale or commercial manufacturing procedures.

The device of U.S. Pat. No. 3,416,530 is manufactured with a plurality of capillary openings that communicate between the exterior of the device and the interior chamber generally defined from a polymeric membrane. While the capillary openings in this construction are effective for releasing certain drugs to the eye, they add considerable complexity to the manufacture of the device because it is difficult to control the size of these openings in commercial manufacturing using various polymers.

U.S. Pat. No. 3,618,604 describes a device that does not involve such capillary openings, but instead provides for the release of the drug by diffusion through a polymeric membrane. The device, as disclosed in a preferred embodiment, comprises a sealed container with the drug contained in an interior chamber. Nonetheless, as described in U.S. Pat. No. 4,014,335, certain problems have been identified with such devices such as the difficult task of sealing the margins of the membrane to form the container. In addition, stresses and strains introduced into the membrane walls from deformation during manufacturing of those devices may cause the reservoir to rupture and leak.

U.S. Pat. No. 6,001,386 to Ashton, et al relates to an implantable sustained release drug implant with an inner core containing an effective amount of a low solubility agent covered by a non-bioerodible polymer coating layer that is permeable to the low solubility agent disclosed.

The above described systems and devices are intended to provide sustained release of drugs effective in treating patients at a desired local or systemic level for obtaining certain physiological or pharmacological effects. However, there are many problems associated with their use, including the fact that it is often difficult to inject the drug implant into the eye which must then be secured in place, for example, via a “suture tab” connected to the drug implant. Injection devices prior to the present invention have not met with great success in being able to quickly deliver the drug implant to its intended target location. For example, maintaining close tolerances between the injector and drug implant, as well as creating sufficient lubricity in the injector cannula to allow a smooth passage of the drug implant therethrough, are a few of the many consistent problems in these prior injector designs.

There thus remains a need for an improved injection device for delivering a drug implant adapted for sustained release of a drug to a patient to obtain a desired local or systemic physiological or pharmacological effect.

### SUMMARY OF THE INVENTION

The present invention addresses the problems associated with prior art injection devices by providing an injection device having an injection needle having a lateral opening formed in the outer surface thereof adjacent the distal, pointed end of the needle. The opening is configured to accept and releasably hold a drug implant therein. The drug implant may be in any of a variety of forms, for example, round, cylindrical, rectangular, square, etc., having a sustained release drug contained within a protective sheath which is designed for slow release of the drug at the implant site, some examples of which may be seen in copending application numbers ______ which are commonly assigned to applicant herein, the entire references of which are incorporated herein by reference.

With the drug implant releasably secured in the lateral opening of the needle, the distal end of the needle is advanced through the tissue (e.g., intravitreal) until it reaches the target site in the organism (e.g., adjacent the retina of a human eye). Various release mechanisms are disclosed herein for releasing the drug implant from the needle opening at the implant site. Once the drug implant is released from the needle, the needle may be withdrawn from the organism. It will thus be appreciated that the drug implant does not travel through a cannula with the present invention. Subsequent steps for securing the drug implant in place, if needed, may be carried out in any known surgical manner (e.g., using sutures).

### BRIEF DESCRIPTION OF THE DRAWINGS

FIG. 1 is a fragmentary, perspective view of the inventive needle with a drug implant shown in spaced relation to the opening in the needle;

FIG. 2 is a side elevational view of FIG. 1;

FIG. 3 is the view of FIG. 2 showing the drug implant positioned within the needle opening and a movable covering;

FIG. 4 is the view of FIG. 3 showing the covering in place over the drug implant and needle opening;

FIG. 5 is a cross-sectional view showing a first embodiment of a securing and release mechanism for the drug implant;

FIG. 6 is a cross-sectional view of a second embodiment showing a friction-fit between the drug implant and needle opening;

FIG. 7 is a cross-sectional view thereof as taken generally along the line 7-7 of FIG. 6;

FIG. 8 is a fragmentary, cross-sectional view of a third embodiment of a securing and release mechanism for the drug implant;

FIG. 9 is the view of FIG. 8 showing the securing and release mechanism in the secured position;

FIG. 10 is a fourth embodiment of a securing and release mechanism for the drug implant;

FIG. 11 is the view of FIG. 10 showing the releasing movement of the mechanism;

FIG. 12 is a fifth embodiment of a securing and release mechanism for the drug implant showing the mechanism in the secured position; and

FIG. 13 is the view of FIG. 12 showing the mechanism in the release position.

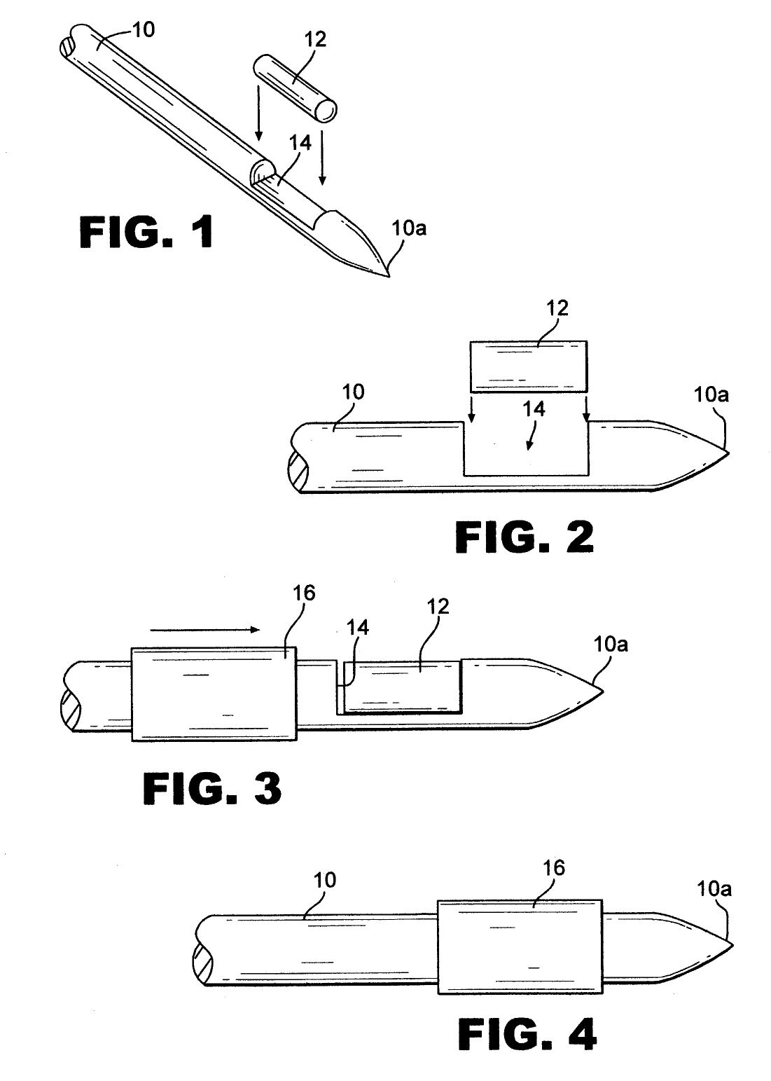

### DETAILED DESCRIPTION OF PREFERRED EMBODIMENTS

Referring now to the drawing, there is seen in a distal end of a needle 10 having a pointed, distal end 10a for directing into the tissue of a target surgical site of a mammalian organism, for example a human eye. The opposite, proximal end of the needle may be of any desired configuration for manual or machine manipulation of needle 10 consistent with the intended uses of needle 10 set forth herein (not shown).

Needle 10 may be made of any suitable material for injection within a mammalian organism, some examples of which include titanium, stainless steel, ceramic, and polymer. The diameter of needle 10 is sized to enable direct injection of needle 10 within the delicate tissues of a human eye.

A drug implant is indicated by reference numeral 12 and is illustrated herein in the shape of a cylinder having an outer diameter less than the diameter of needle 10 adjacent distal end 10a thereof, although the drug implant may be of other shapes as desired. Drug implant 12 is a slow-release implant capable of controlled release of a drug to the implant site. Needle 10 is the vehicle for delivering implant 12 to the implant site.

More particularly, needle 10 is seen to include a lateral opening 14 therein adjacent distal end 10a thereof. Opening 14 serves to releasably hold drug implant 12 therein until the distal end 10a of needle 10 reaches the implant site in the organism, at which time drug implant 12 is released from opening 14. Once drug implant 12 is released at the implant site, needle 10 may be withdrawn from the organism and either sterilized for subsequent use or discarded in single-use designs of needle 10.

Opening 14 may be of any desired configuration, but is preferably configured to match the shape of the drug implant 12 releasably held therein. As seen in FIGS. 3 and 4, a protective cover 16 may be provided in coaxial, sliding engagement with needle 10. Covering 16 is selectively movable along the shaft of needle 10 from the open position seen in FIG. 3 to the closed position seen in FIG. 4 wherein drug implant 12 is prevented from falling free of opening 14 until the target site is reached, at which time covering 16 may be moved in the opposite direction to the open position. An extensible rod (not shown) may be attached to covering 16 to selectively move it between the open and closed positions.

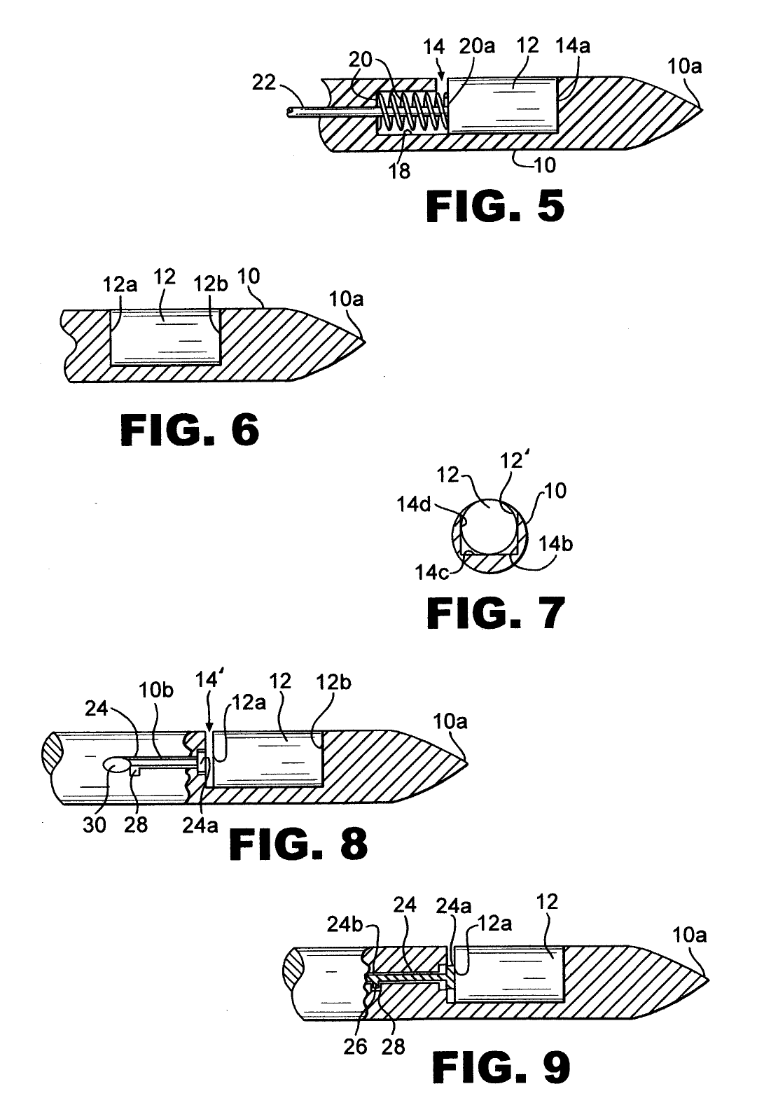

Attention is now turned to FIG. 5 which shows a first embodiment of an implant release mechanism. In this embodiment; a center bore 18 is provided as an axial extension of opening 14 wherein a spring 20 may be positioned to apply a biasing force against drug implant 12 such that implant 12 is forcibly held between the spring and the distal end wall 14a of opening 14. A longitudinally extending rod 22 may be attached to the distal end 20a of spring 20 whereby rod 22 may be retracted in a direction opposite distal needle end 10a to release the biasing force of spring 20, thereby allowing release of implant 12 from needle 10 at the implant site.

FIG. 6 shown a second embodiment wherein drug implant 12 is engaged in opening 14 by friction-fit at opposite ends 12a,12b thereof. As seen in the cross-section view of FIG. 7, drug implant 12 which is cylindrical in this embodiment, is engaged in opening 14 by the three planar walls 14b, 14c, and 14d of opening 14 frictionally engaging the cylindrical side wall 12′ thereof. Although no release mechanism is shown in FIGS. 6 or 7, any of the release mechanisms described herein may be used in combination with any of the securing mechanisms described herein which will be detailed more fully below.

FIGS. 8 and 9 show another embodiment of a securing and release mechanism for implant 12. To load the implant 12 in opening 14, longitudinally extending rod 24 is retracted to the position shown in FIG. 8 whereby a clearance 14′ is provided in opening 14 to allow easy insertion of implant 12 therein. Once implant 12 is inserted into opening 14, rod 24 may be moved in the opposite direction toward distal end 10a until the distal end of the rod 24a engages the proximal end 12a of the implant 12. Rod 24 and the axial bore 10b in which rod 24 extends may be provided with latch 26 and slot 28, respectively, such that rod 14 may be locked into the engaged position seen in FIG. 9 until the implant site is reached, at which time latch 26 may be disengaged from slot 28 (e.g., by rotating rod 24). A manually operable push-tab 30 may be attached to rod 24 and lie exteriorly of needle 10 to allow easy one-touch manipulation of rod 24.

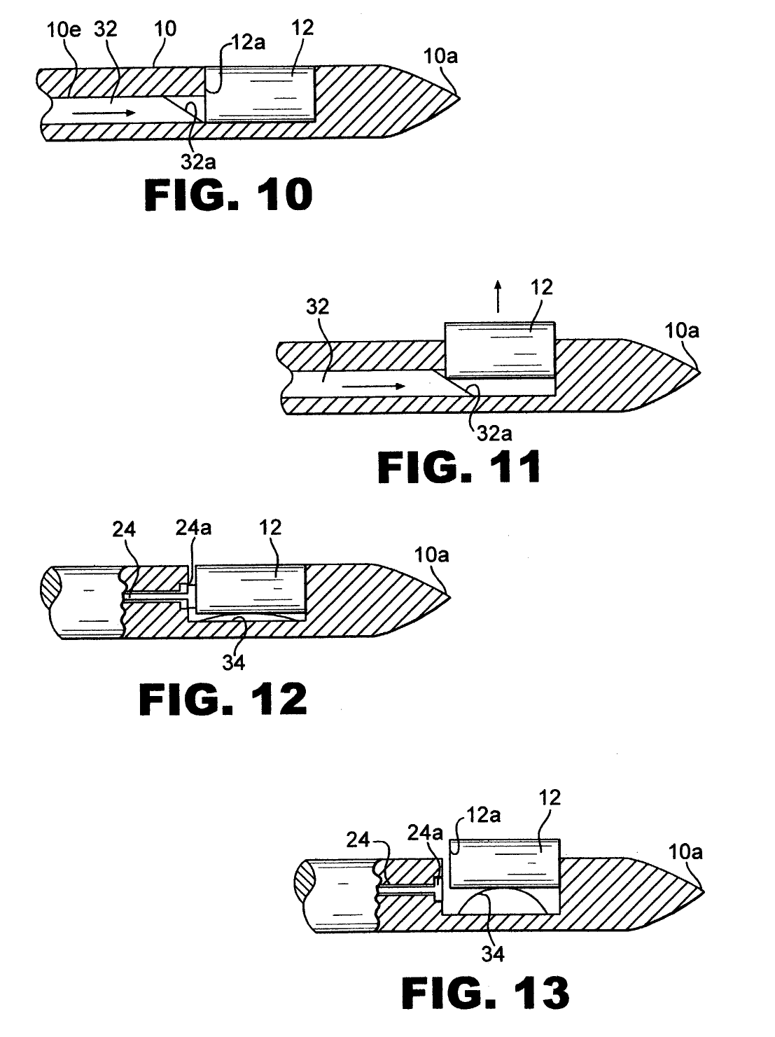

Referring to FIGS. 10 and 11, a release mechanism is shown comprising a shaft 32 reciprocally mounted in axial bore 10e formed in needle 10. Shaft 32 includes an angled distal end 32a which may be moved to the secured position wherein end 32a is in just-touching relation to the proximal end of implant 12 as seen in FIG. 10. To release implant 12 at the implant site, shaft 32 is moved further toward distal end 10a whereupon the leading tip of end 32a wedges beneath implant 12 causing implant 12 to dislodge from opening 14 as seen in FIG. 11.

In yet a further embodiment of implant release mechanism, the rod 24 of the embodiment of FIGS. 8 and 9 is used in combination with a spring 34 located within opening 14. In the load/release position shown in FIG. 13, spring 34 is unbiased and rod 24 is retracted. In this position, implant 12 may be inserted into opening 14 and pressed against spring 34 to put spring 34 in tension. In the fully inserted position of implant 12 seen in FIG. 12, spring 34 is fully tensioned and applying an outward biasing force against implant 12. While the implant and spring are held in this position (e.g., by pressing with a finger), rod 24 is extended until end 24a thereof is engaged against the proximal end 12a of implant 12. Rod 24 may be locked in place as explained above with regard to FIGS. 8 and 9, at which time implant 12 is held tightly within opening 14 since the force of end 24a against implant 12 is stronger than the biasing force of spring 24. Once needle 10 has been injected within the organism and end 10a thereof is at the implant site, the user releases and retracts rod 24 to the position seen in FIG. 13, at which time the force against implant end 12a is removed and the biasing force of spring 34 takes over to force implant 12 from opening 14. Needle 10 may then be retracted from the implant site, leaving implant 12 in place.

It will thus be appreciated that the present invention provides an improved injection device for delivering slow-release drug implants to the inside body of an organism. Since the drug implant is both held and released at the distal end of the needle, there is no need for a cannula delivery of the implant which obviates that many problems associated therewith as explained in the Background section hereof.

What is claimed is:

1. A device for delivering a drug implant to an implant site within a mammalian body, said device comprising:
a) a needle having a distal, pointed end for directing into the tissues of the mammalian body, said needle further including a lateral opening adjacent said distal end, said lateral opening sized to releasably hold a drug implant therein;
b) a drug implant holder for holding said drug implant in said opening; and
c) a drug implant release mechanism for selectively releasing said drug implant from said opening at said implant site.

2. The device according to claim 1, wherein said drug implant holder comprises a rod having opposite distal and proximal ends, said needle further including an axial bore in communication with said opening, said rod mounted for reciprocal movement within an axial bore such that said rod may be selectively moved between an extended position wherein said distal end thereof engages said drug implant when said drug implant is in said opening, and a retracted position wherein said distal end of said rod is disengaged from said drug implant whereby said drug implant may be withdrawn from said opening at said implant site.

3. The device according to claim 2 wherein said rod may be releasably locked in said extended position.

4. The device according to claim 2 wherein said drug implant release mechanism comprises a spring located within said opening, said spring positioned to provide a biasing force against said drug implant in a direction outward of said opening.

5. The device according to claim 1 wherein said drug holder is a spring positioned within said needle with said spring applying a biasing force against said drug implant in the direction of said needle distal end.
